(19) United States
(12) Patent Application Publication
(10) Pub. No.: US 2026/0165954 A1
(43) Pub. Date: Jun. 18, 2026

(54) BIODEGRADABLE FUSE-AND-RESERVOIR SYSTEM FOR THE PULSED DELIVERY OF BIOACTIVE AGENTS

(71) Applicant: University of Mississippi, University, MS (US)

(72) Inventors: Glenn M. WALKER, University, MS (US); Thomas Anthony WERFEL, Oxford, MS (US)

(21) Appl. No.: 19/129,443

(22) PCT Filed: Oct. 30, 2023
(86) PCT No.: PCT/US2023/078162

§ 371 Date: May 13, 2025

Related U.S. Application Data
(60) Provisional application No. 63/425,344, filed on Nov. 15, 2022.

(51) Int. Cl. A61K 9/00; A61K 45/06; A61K 47/38

(52) U.S. Cl. CPC A61K 9/0024; A61K 45/06; A61K 47/38

(57) ABSTRACT

Disclosed herein is a device for delivering one or more bioactive agents, the device comprising a first layer comprising a first biodegradable polymer, wherein the first layer has a first and a second side, and wherein a plurality of reservoirs comprising one or more bioactive agents are present on the first side; a second layer comprising a second biodegradable polymer, wherein the second layer is adjacent to the first side of the first layer; and a third layer comprising a third biodegradable polymer, wherein the third layer encapsulates the first and second layers, and wherein at least one face of the device comprises an exposed second layer not coated by the third biodegradable polymer. In one aspect, the first and third biodegradable polymers are poly-ε-caprolactone, while the second biodegradable polymer is a mixture of cellulose acetate phthalate and a poloxamer. Also disclosed are methods for fabricating the devices.

### CROSS-REFERENCE TO RELATED APPLICATIONS

This application claims the benefit of U.S. Provisional Application No. 63/425,344, filed on Nov. 15, 2022, which is incorporated herein by reference in its entirety.

### STATEMENT REGARDING FEDERALLY SPONSORED RESEARCH OR DEVELOPMENT

This invention was made with government support under grant number R21EB031454-01A1 awarded by the National Institutes of Health. The government has certain rights in the invention.

### BACKGROUND

Most implantable drug delivery devices continuously release a drug (e.g., birth control implants). However, for some applications such as, for example, cancer, certain hormone disorders, and the like, periodic, or pulsatile, drug release is more desirable. The known commercial pulsatile release device from Microchips Biotech is expensive and large (4.5 cm×5.5 cm) and requires a surgical procedure for implantation. Following delivery of all doses, this same device must be removed. For treatments that last months or years, multiple surgical procedures would be required, increasing potential risks to the patient.

Delivery devices capable of delivering bioactive agents over several months is an attractive approach for treating certain types of disease states. For example, depression is a major public health burden domestically and worldwide. Treatment options for patients with major depressive disorder (MDD) consist primarily of psychotherapy and pharmacotherapy. In the latter category, specifically, there have been only incremental advances in treatment options until recently when Esketamine, a form of ketamine given as a nasal spray, was approved.

Primary challenges to the treatment of MDD are the relatively low response rates to medication as well as high relapse in a large subset of patients. The most comprehensive study of MDD undertaken was the National Institute of Mental Health-funded Sequenced Treatment Alternatives to Relieve Depression (STAR*D) trial. The trial outlined an algorithmic, sequential treatment approach and thus allows for an estimated likelihood of antidepressant success with subsequent trials. Acute remission rates declined with each additional trial (trial 1, 37%; trial 2, 31%; trial 3, 14%; trial 4, 13%). Correspondingly, the probabilities of remitting and maintaining remission for 1 year decrease with each additional trial (26% for level 1, 14% for level 2, 5% for level 3, and 3% for level 4). Unfortunately, this corresponds to over 43% of patients who fail the first two trials.

Microdosing is one approach for the treatment of depression and other diseases; however, this approach also suffers from limitations. For example, a major hurdle to furthering the research on 5HT_{2A} agonists such as LSD and psilocybin for the treatment of depression is their current status as Schedule I drugs. Given the potential concerns around diversion or abuse of these compounds, as well as the strict controls on their distribution, dosing is limited to monitored settings. This requires patients to come in for dosing on an every-other (Q48) or every-third day (Q72) schedule for several weeks, which will likely limit patient participation and treatment.

What is needed is devices enabling the pulsed delivery of bioactive agents to patients such that the patients do not need to be responsible for taking the bioactive agent, which is instead delivered automatically at desired intervals to the patient. An ideal device and/or approach would further not require repeated clinic visits by patients, thereby increasing patient compliance and achieving desired treatment outcomes. Additionally, the device would be biodegradable so surgical removal would not be required, and ideally the device could be administered via a single injection. These needs and other needs are satisfied by the present disclosure.

### SUMMARY

In accordance with the purpose(s) of the present disclosure, as embodied and broadly described herein, the disclosure, in one aspect, relates to a device for delivering one or more bioactive agents to a subject, the device comprising (a) a first layer comprising a first biodegradable polymer, wherein the first layer has a first side and a second side, and wherein a plurality of reservoirs comprising one or more bioactive agents are present on the first side of the first layer; (b) a second layer comprising a second biodegradable polymer, wherein the second layer is adjacent to the first side of the first layer; and (c) a third layer comprising a third biodegradable polymer, wherein the third layer encapsulates the first layer and the second layer, and wherein at least one face of the device comprises an exposed second layer not coated by the third biodegradable polymer. In one aspect, the first and third biodegradable polymers are poly-ε-caprolactone (PCL), polysebacic acid, polylactic acid, or any combination thereof, and the second biodegradable polymer is a mixture of cellulose acetate phthalate (CAP) and a poloxamer. Also disclosed are methods for fabricating the devices.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show the assembly and structure of a microfuse device described herein.

FIG. 2 shows the direct correlation established between polymer mass and resulting film thickness (with volume kept constant).

FIGS. 3A-3C show rhodamine B dye release from eroding polymer films. Time of drug release is delayed as film thickness increases. FIG. 3A: Erosion time versus CAPP film thickness. FIG. 3B: Cumulative release of rhodamine from 0.1-0.4 mm films. Error bars are included, but in some cases are not visible due to small error. FIG. 3C: Erosion time for 70:30-90:10 CAPP films at each thickness (0.1, 0.2, 0.3, and 0.4 mm, respectively).

FIGS. 4A-4D show an overview of a mold-making process according to the present disclosure. FIG. 4A is a schematic of a stamp design. FIG. 4B is a silicon master with features made from an epoxy-based photoresist (SU-8). FIG. 4C shows a polydimethylsiloxane (PDMS) replica of the silicon master. FIG. 4D shows an epoxy resin mold made from the master shown in FIG. 4C.

FIG. 5A shows a representative polycaprolactone/polyvinyl alcohol (PCL/PVA) film ready for hot embossing by the mold shown in FIG. 4D. FIG. 5B shows an example of the first layer of a hot-embossed microfuse device according to one embodiment of the present disclosure.

FIG. 6A shows an SEM image of a cellulose acetate phthalate-pluronic F-127 (CAPP)-coated fluorescein (FITC)-loaded device. FIG. 6B shows an SEM image of a single drug-loaded reservoir. FIG. 6C shows an image of PCL-encapsulated and CAPP-coated FITC-loaded microdevices.

FIG. 7 shows a preliminary fluorescein release curve. Fluorescence intensity over time reveals that all devices display a pulsed release profile and 5 of 6 devices demonstrate the same timing of release.

FIG. 8 shows a cross-sectional view of a disclosed device in operation. One end is uncoated, allowing access to the at least one second biodegradable polymer by the surrounding environment. Surface erosion of the at least one second biodegradable polymer sequentially exposes each reservoir, in turn, allowing for drug release.

FIGS. 9A-9B show a microfuse device design according to one embodiment of the present disclosure. (FIG. 9A) Cross-section of a microfluidic fuse. (FIG. 9B) Side view of a microfabricated device for long-term pulsatile drug release showing the μfuse concept. The proposed approach results in a smaller implantable device with more drug reservoirs than previously reported devices.

FIGS. 10A-10F show fabrication and structural characterization of μfuse-based device (MDIs). (FIG. 10A) Process schematic overviewing the soft-lithography of μfuse-based MDIs. Representative images of (FIG. 10B) Stamp, (FIG. 10C) Stamped PCL Base, (FIG. 10D) FITC-filled PCL, (FIG. 10E) Cross-section (top) and side view (bottom) of CAPP fuse laid over FITC-filled PCL, and (FIG. 10F) Cross-section of Final Trimmed MDI at a reservoir (right).

FIG. 11 shows precise interval drug release from μfuse-based MDIs. MDIs fabricated by the process laid out in FIGS. 10A-10F reliably re-lease FITC pulses every three days when incubated in 40% FBS.

FIGS. 12A-12G show in vivo delivery of 5HT2A agonist DOI from single- and multi-layer CAPP films. (FIG. 12A) In vitro cumulative release of DOI from 0.1 mm CAPP films. (FIG. 12B) Scheme of subcutaneous implantation of 0.1 mm DOI-loaded CAPP films and experimental time course. Pharmacokinetics of DOI released from subcutaneously implanted, single-layered CAPP films in (FIG. 12C) plasma (n=4) and (FIG. 12D) brain (n=4). (FIG. 12E) Scheme of subcutaneous implantation of multilayered CAPP films and experimental time course. Pharmacokinetics of DOI released from multilayered CAPP films in (FIG. 12F) plasma (n=4), and (FIG. 12G) brain (n=4).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a bioactive agent,” “a polymer,” or “a time interval,” includes, but is not limited to, mixtures, combinations, or ranges of two or more such bioactive agents, polymers, or time intervals, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a poly(lactic-co-glycolic acid) (PLGA) refers to an amount that is sufficient to achieve the desired improvement in the property modulated by the formulation component, e.g. achieving the desired release timing of a bioactive agent as a reservoir lid made from PLGA dissolves. The specific level in terms of wt % in a composition required as an effective amount, or thickness of a layer containing the same, will depend upon a variety of factors including the amount of PLGA in the reservoir lid, the amount and type of other polymers present in the lid, and the desired bioactive agent dosing level and timing.

A “microfuse” or “μfuse” as used herein refers to a disclosed surface-eroding microfluidic fuse device for pulsed delivery of pharmaceutical agents to a subject. In one aspect, the μfuse device is made from or includes biodegradable polymers and can be implanted in a subject.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Delivery Devices

Described herein are devices that provide the pulsed delivery of a bioactive agent to a subject upon administration of the device to the subject. The devices are composed of biodegradable polymers such that when administered to the subject the second layer polymer erodes and releases the bioactive agent at specific time intervals. The term “microdosing” is referred to as the administration of a bioactive agent in small quantities (e.g., microgram scale) every 3-7 days to treat or prevent a disease or symptom thereof. In some aspects, the second layer can further incorporate an additional bioactive agent that should be released at a constant rate over time, separately from the bioactive agent contained in the reservoirs.

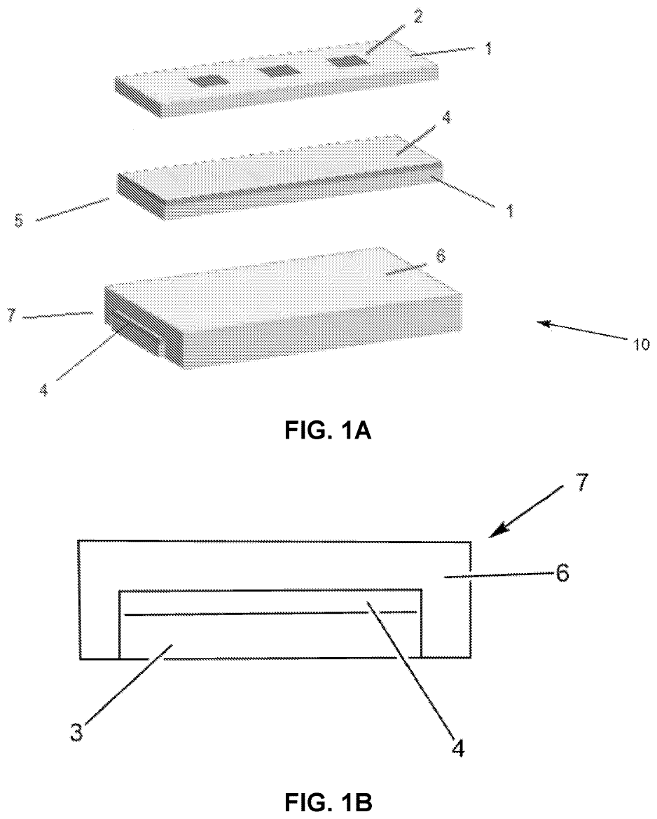

An example of a delivery device described herein is provided in FIGS. 1A-1B, which is referred to herein as a microfuse 10. Referring to FIG. 1A, a first layer 1 composed of a first biodegradable polymer has a plurality of reservoirs 2 comprising the bioactive agent. The number, shape, and dimensions of the reservoirs can vary depending upon the amount of bioactive agent to be delivered to the subject. A second layer 4 comprising at least one second biodegradable polymer is applied to the side of the first layer with the reservoirs so that the second layer is adjacent to (i.e., in contact with) the first side 3 of the first layer to produce layered system 5. Finally, the layered system 5 is encapsulated with a third biodegradable polymer to produce the device 6. The layered system 5 is encapsulated such that all but one surface of the layered system is covered with the third biodegradable polymer. In an aspect, the device has a longitudinal axis, parallel to which the reservoirs are arranged in one or more rows, and the one surface of the layered system that is not covered with the third biodegradable polymer can be a face that is perpendicular to the longitudinal axis, thus exposing a portion of the second layer to the surrounding medium. In some aspects, the longitudinal axis will extend the length of, and be parallel to, the longest side of the disclosed devices. In an aspect, when the reservoirs are arranged in parallel rows, each row of reservoirs can incorporate a different bioactive agent, thus allowing the devices to release two drugs simultaneously.

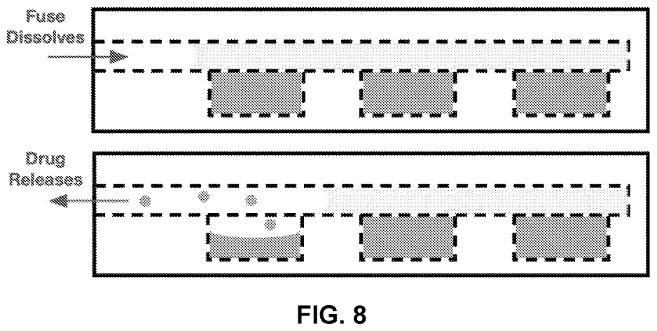

A schematic of a disclosed device in use is provided in FIG. 8. In one aspect, the third layer coating is not present at one face perpendicular to the longitudinal axis of the device (left-hand side of top image in FIG. 8), exposing the at least one second biodegradable polymer. When the second biodegradable polymer has eroded to the point that a reservoir is exposed (see bottom image in FIG. 8), the drug contained in that reservoir is released.

In one aspect, the device further includes a plurality of distances between reservoirs, where the plurality of distances are measured parallel to the longitudinal axis. In a further aspect, each distance of the plurality can be the same. In an alternative aspect, at least one of the plurality of distances can be different from at least one other of the plurality of distances. In a still further aspect, equal distances between reservoirs can lead to equal times between drug release, while different distances between reservoirs can lead to longer or shorter times between instances of drug release, where a long distance between reservoirs would mean less frequent drug release and a short distance between reservoirs would mean more frequent drug release.

In any of these aspects, the overall length of the devices (i.e., the dimension parallel to the longitudinal axis) is not limited. In one aspect, a longer device may incorporate more reservoirs, or a greater distance between reservoirs, or both, in order to tailor the number of doses and timing of doses to a particular application.

In one aspect, the exposed second layer erodes from the surface only and does not bulk erode. In a further aspect, although not all polymers are surface-eroding, the second layer is composed of a polymer that is capable of surface erosion. The surface-eroding polymer is further characterized below. In any of these aspects, the surface-eroding dissolvable polymer layer can be fabricated with different distances between reservoirs to enable controlled release timing for bioactive agents in different reservoirs of the plurality, as discussed further below.

In one aspect, each reservoir can have the same volume. In an alternative aspect, each reservoir can have a different volume. For example, in one aspect, it may be desirable to have a higher initial dose of a drug, followed by lower subsequent doses, or it may be desirable to taper amount of drug in the body by delivering successively smaller doses over time.

In one aspect, each one of the plurality of reservoirs contains a different bioactive agent. In another aspect, each one of the plurality of reservoirs contains the same bioactive agent. In still another aspect, combinations of these possibilities are envisioned—for example, two reservoirs containing the same bioactive agent and a third reservoir containing a different bioactive agent, and the like. Further in this aspect, since the second layer surface erodes, a reservoir with a greater distance from the exposed surface will result in a later release time for the bioactive agent contained in the reservoir, while a reservoir with a shorter distance from the exposed surface will result in an earlier release time for the bioactive agent contained in the reservoir. In one aspect, in this manner, 2, 3, 4, or more reservoirs, each having a different distance from the exposed surface, can be included in the same device such that delivery of the bioactive agents contained therein is pulsed or delivered at regular intervals, without requiring any additional action from a patient or clinician. Also contemplated are rows of reservoirs containing different bioactive agents but the same distance from the exposed surface, thus allowing for two or more bioactive agents to be released simultaneously. In an alternative aspect, the rows of reservoirs can be covered by or adjacent to different second layer polymers, where the different second layer polymers erode at different rates, allowing for different release timing for each row. In another aspect, the rows of reservoirs can be covered by the same second layer polymer, having the same or a different distance from the exposed surface as outlined previously.

The selection of the at least one second biodegradable polymer as well as the distance of the reservoir from the exposed surface will determine the release rate of the one or more bioactive agents, as the second polymer layers erodes (i.e., biodegrades) over time after the device is administered to the subject. In one aspect, first biodegradable polymer and the third biodegradable polymer are the same or a different polymer. Further in this aspect, by making the first and third layers biodegradable, the delivery device will not need to be removed from the body after it is finished dispensing doses. In another aspect, the first biodegradable polymer and the at least one second biodegradable polymer are different.

In some aspects, the at least one second biodegradable polymer can be mixed with or otherwise include a bioactive agent. In some aspects, this bioactive agent can be the same as one of the one or more bioactive agents in the reservoirs, or can be a different bioactive agent.

In one aspect, the first layer 1 of the device as depicted in FIG. 1A is composed of a polymer that biodegrades at a rate less than that of the polymer in the second layer 4. In another aspect, the first layer 1 of the device as depicted in FIG. 1A is composed of a polymer that biodegrades at a rate that is at least two times, three times, or four times less than that of the at least one second biodegradable polymer in the second layer 4. In one aspect, the first biodegradable polymer is poly-ε-caprolactone (PCL), polysebacic acid, polylactic acid, a polyester, a polyanhydride, a polyurethane, a polyphosphazine, a polyalkylcyanoacrylate, a polypeptide, a block copolymer comprising polyethylene glycol (PEG), or any combination thereof. In another aspect, the first layer has a thickness of from about 0.1 mm to about 2 mm, or of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or about 2 mm, or a combination of any of the foregoing values, or a range encompassing any of the foregoing values, where any value can be the upper or lower endpoint of the range.

In one aspect, the at least one second biodegradable polymer in the second layer 4 is a polymer that biodegrades faster than the first polymer in the first layer 1. In one aspect, the at least one second biodegradable polymer can include a mixture of two or more different polymers. In one aspect, the at least one second biodegradable polymer comprises a mixture of cellulose acetate phthalate (CAP) and a poloxamer, a polyanhydride, a polyorthoester, or any combination thereof. In one aspect, CAP has a molecular weight of from about 1,000 g/mol to about 5,000 g/mol, or about 2,000 g/mol to about 3,000 g/mol.

In one aspect, the poloxamer is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

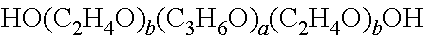

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from 2,000 to 15,000, 3,000 to 14,000, or 4,000 to 12,000. Poloxamers useful herein are sold under the tradename Pluronic® manufactured by BASF. Non-limiting examples of poloxamers useful herein include, but are not limited to, those in Table 1. In one aspect, the poloxamer is F-127

TABLE 1
Non-Limiting Examples of Poloxamers Useful Herein

| | | Average | Average | |
|---|---|---|---|---|
| | | number | number | CMC |
| Copolymer | MW | of EO units | of PO units | (M) |
| F68 | 8,400 | 152.73 | 28.97 | 4.8 × 10⁻⁴ |
| P103 | 4,950 | 33.75 | 59.74 | 6.1 × 10⁻⁶ |
| P105 | 6,500 | 73.86 | 56.03 | 6.2 × 10⁻⁶ |
| P123 | 5,750 | 39.2 | 69.4 | 4.4 × 10⁻⁶ |
| F127 | 12,600 | 200.45 | 65.17 | 2.8 × 10⁻⁶ |
| L121 | 4,400 | 10.00 | 68.28 | 1.1 × 10⁻⁶ |

In one aspect, wherein the at least one second biodegradable polymer includes a mixture of cellulose acetate phthalate (CAP) in the amount of about 50 mol % to about 90 mol % and a poloxamer in the amount of about 10 mol % to about 50 mol %. In another aspect, the at least one second biodegradable polymer includes cellulose acetate phthalate (CAP) in the amount of about 50 mol %, 55 mol %, 60 mol %, 65 mol %, 70 mol %, 75 mol %, 80 mol %, 85 mol %, or 90 mol %, where any value can be a lower and upper endpoint of a range (e.g., 60 mol % to 80 mol %, etc.). In another aspect, the at least one second biodegradable polymer includes a poloxamer in the amount of about 10 mol %, 15 mol %, 20 mol %, 25 mol %, 30 mol %, 35 mol %, 40 mol %, 45 mol %, or 50 mol %, where any value can be a lower and upper endpoint of a range (e.g., 20 mol % to 40 mol %, etc.). In one aspect, the second layer 4 has a thickness of from about 0.1 mm to about 2 mm, or of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or about 2 mm, or a combination of any of the foregoing values, or a range encompassing any of the foregoing values, where any value can be the upper or lower endpoint of the range. In one aspect, the second layer 4 is a surface-erodible polymer network with known erosion rate of about 1 μm/h to about 10 μm/h, or at about 1, 2, 3, 4, 5, 6, 7, 8, 9, or about 10 μm/h, or a combination of any of the foregoing values, or a range encompassing any of the foregoing values, where any value can be the upper or lower endpoint of the range.

The device as depicted in FIGS. 1A-1B can be produced using the following non-limiting process. In one aspect, the biodegradable polymer poly-ε-caprolactone (PCL) will form the main body of the device which houses the drug and fuse (i.e., the first and third layers). Drug reservoirs will be fabricated in the first layer by hot embossing via a Carver heated press and a microfabricated stamp. PCL can be molded and cast using relatively low temperatures (e.g., melting point of 60° C.) without damaging bioactive agents with high heat. In addition, the first and third polymer will degrade slowly in vivo and create devices that can last over one year in the body.

After hot embossing the first layer 1, powdered bioactive agent can be packed into the reservoirs 2 using the Carver press without heating. This approach to loading the drug reservoirs is a particularly attractive feature, because it can be used with a variety of bioactive agents and does not rely on heat or solvents for loading. Next, the loaded devices will be inverted and very briefly dipped into a solution of the at least one second biodegradable polymer (e.g., CAP and poloxamer) dissolved in a solvent such as, for example, acetone. The solvent will rapidly evaporate leaving a thin film of the at least one second biodegradable polymer that seals the drug reservoirs and forms a fuse. Multiple dips can be performed to adjust the thickness of the second polymer layer 4 based on the application. Finally, the third biodegradable polymer (e.g., PCL) will be melted and poured over the layered system 5, encapsulating the layered system. The final device will be cut out and ready for use.

After the cutting step above is performed, the second layer 4 is exposed on one or more side of the device. Referring to FIG. 1B, the face 7 has an exposed second layer 4. The exposed second layer 4 at face 7 acts as a fuse. As the fuse dissolves along the exposed face, the reservoirs containing the bioactive agent are periodically exposed to the intracellular space and release their contents. The distance between reservoirs 2 under the microfluidic fuse (i.e., second layer 4) controls the release schedule, and the spacing is determined at the time of manufacture and can be tailored to the specific application. Although FIGS. 1A-1B depict a single fuse (i.e., single second layer 4), multiple fuses on a single device, each with a unique erosion rate, can be used to permit very long-term implantable drug release. In other aspect, multiple fuses could be used for the timed release of incompatible drugs and/or response to variety of environmental cues.

The devices described herein and the methods for producing the same permit the inclusion of a variety of different types of bioactive agents. In one aspect, the bioactive agent includes an antibiotic, a pain reliever, an immune modulator, a growth factor, an enzyme inhibitor, a hormone, a messenger molecule, a cell signaling molecule, a receptor agonist, an oncolytic virus, a chemotherapy agent, a receptor antagonist, a nucleic acid, a nanoparticle, a drug conjugate, an antibody-drug conjugate, or any combination thereof.

In another aspect, the bioactive agent includes a 5HT_{2A} agonist such as, for example, 2,5-dimethoxy-4-iodoamphetamine (DOI), 1-acetyl-N,N-diethyllysergamide (ALD-52), O-acetylpsilocin (4-AcO-DMT), lysergic acid diethylamide (LSD), or psilocybin. The devices as described herein are capable of delivering consistent, long-term microdoses of the 5HT_{2A} agonist that will overcome major barriers of compliance, cost, and abuse. The microfuse devices described herein are an efficacious and safe way to perform microdosing by delivering the bioactive agent about every 48 hours to about 96 hours, or about 72 hours over many months.

The devices described herein can be administered to a subject using techniques known in the art. In one aspect, the device is implanted in the subject. In one aspect, a clinician can subcutaneously inject the device instead of performing a minor surgical procedure. This streamlined implantation procedure will save time and money and increase patient compliance even further. In another aspect, the devices described herein can be formulated with a pharmaceutically-acceptable excipient suitable for injection.

### Stimulus-Responsive Devices

Also contemplated herein are stimulus-responsive devices. In a further aspect, a stimulus-responsive device according to the present disclosure would degrade only if a specific type of molecule were present in the surrounding fluid. In one aspect, a reactive oxygen species (ROS)-responsive device could be made from a polysulfide or polythioketal. In another aspect, a reduction-responsive device could be made from disulfide linked polymers, an acid-cleavable device from acid-cleavable polymers such as, for example, ketals or imines, or an enzymatically cleavable device made from enzymatically cleavable polymers such as, for example, enzyme-cleavable peptide cross links.

### Methods of Fabricating the Delivery Devices

In one aspect, disclosed herein is a method for making the disclosed devices, including at least the steps of:
(a) fabricating a mold defining a negative shape of a plurality of devices;
(b) depositing the first biodegradable polymer in the mold to create the first layer;
(c) loading the bioactive agent in at least one of the plurality of reservoirs to create a plurality of loaded device substrates;
(d) depositing the at least one second biodegradable polymer on top of the plurality of loaded device substrates to create a second layer that is attached to the first layer and create a plurality of loaded devices; and
(e) encapsulating the plurality of loaded devices with the third biodegradable polymer to create the third layer, wherein the third layer encapsulates the first layer and the second layer, and wherein at least one face of each one of the plurality of the devices comprises an exposed second layer not coated by the third biodegradable polymer.

In any of these aspects, “depositing” can refer to any method for incorporating a polymer into the devices, including, but not limited to, hot embossing, 3D printing, spray coating, spin coating, dip coating, vacuum deposition, or any other method known in the art.

In some aspects, in step (b), the first biodegradable polymer is deposited hot embossing, injection molding, or 3D printing with a biodegradable resin. In some aspects, in step (d), the at least one second biodegradable polymer can be spin-coated, spray-coated, or hot embossed to create the second layer.

Exemplary materials and processes useful for fabricating the devices are provided in the Examples.

### 3D Printing

In one aspect, a 3D printing process can be used instead of the disclosed hot embossing process. In an alternative aspect, a 3D printing process can be used in combination with other techniques and polymers disclosed herein. In one aspect, some biocompatible polymers suitable for 3D printing may have different release profiles for bioactive agents, and layer thickness, crosslinking or lack thereof, and other methods and parameters can be manipulated to provide devices according to the present disclosure.

In another aspect, any biodegradable monomer, oligomer, or polymer compatible with digital light processing (DLP) 3D printing processes can be used to manufacture all or part of the disclosed devices. In an aspect, in DLP 3D printing, one or more monomers and/or oligomers are contacted with a photoinitiator and subjected to ultraviolet light, inducing polymerization of the monomers and/or oligomers.

In some aspects, biodegradable fillers can also be used in combination with one or more polymers in the disclosed devices. In a further aspect, the filler can consist largely of starches and can be derived from a common food product such as, for example, potato, sweet potato, and/or yam.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.

Aspect 1. A device for delivering one or more bioactive agents to a subject, the device comprising
a first layer comprising a first biodegradable polymer, wherein the first layer has a first side and a second side, and wherein a plurality of reservoirs comprising the one or more bioactive agents are present on the first side of the first layer;
a second layer comprising at least one second biodegradable polymer, wherein the second layer is adjacent to the first side of the first layer;
a longitudinal axis; and
a third layer comprising a third biodegradable polymer, wherein the third layer encapsulates the first layer and the second layer, and wherein at least one face of the device perpendicular to the longitudinal axis comprises an exposed second layer surface not coated by the third biodegradable polymer.

Aspect 2. The device of aspect 1, wherein the first biodegradable polymer and the at least one second biodegradable polymer are different polymers.

Aspect 3. The device of aspect 1 or 2, wherein the first biodegradable polymer and the third biodegradable polymer are the same polymer.

Aspect 4. The device of any one of aspects 1-3, wherein the first biodegradable polymer comprises poly-ε-caprolactone (PCL), polysebacic acid, polylactic acid, a polyester, a polyanhydride, a polyurethane, a polyphosphazine, a polyalkylcyanoacrylate, a polypeptide, a block copolymer comprising polyethylene glycol (PEG), or any combination thereof.

Aspect 5. The device of any one of aspects 1-4, wherein the first layer has a thickness of from about 0.1 mm to about 2 mm.

Aspect 6. The device of any one of aspects 1-5, wherein the at least one second biodegradable polymer erodes from the exposed second layer surface.

Aspect 7. The device of any one of aspects 1-6, wherein the at least one second biodegradable polymer comprises a mixture of cellulose acetate phthalate (CAP) and a poloxamer, a polyanhydride, a polyorthoester, or any combination thereof.

Aspect 8. The device of any one of aspects 1-7, wherein the at least one second biodegradable polymer comprises a mixture of cellulose acetate phthalate (CAP) in the amount of about 50 mol % to about 90 mol % and a poloxamer in the amount of about 10 mol % to about 50 mol %.

Aspect 9. The device of any one of aspects 1-8, wherein the second layer has a thickness of from about 0.1 mm to about 2 mm.

Aspect 10. The device of any one of aspects 1-9, wherein the third biodegradable polymer comprises poly-ε-caprolactone (PCL), polysebacic acid, polylactic acid, a polyester, a polyanhydride, a polyurethane, a polyphosphazine, a polyalkylcyanoacrylate, a polypeptide, a block copolymer comprising polyethylene glycol (PEG), or any combination thereof.

Aspect 11. The device of any one of aspects 1-10, wherein the third layer has a thickness of from about 0.1 mm to about 2 mm.

Aspect 12. The device of any one of aspects 1-11, wherein the one or more bioactive agents comprises an antibiotic, a pain reliever, an immune modulator, a growth factor, an enzyme inhibitor, a hormone, a messenger molecule, a cell signaling molecule, a receptor agonist, an oncolytic virus, a chemotherapy agent, a receptor antagonist, a nucleic acid, a nanoparticle, a drug conjugate, an antibody-drug conjugate, or any combination thereof.

Aspect 13. The device of any one of aspects 1-12, wherein each one of the plurality of reservoirs comprises a different bioactive agent.

Aspect 14. The device of any one of aspects 1-12, wherein each one of the plurality of reservoirs comprises the same bioactive agent.

Aspect 15. The device of any one of aspects 1-14, wherein the plurality of reservoirs are arranged in one or more rows parallel to the longitudinal axis.

Aspect 16. The device of aspect 15, wherein the device comprises at least two rows of reservoirs, and wherein each row of reservoirs comprises a different bioactive agent.

Aspect 17. The device of aspect 15, wherein the device comprises at least two rows of reservoirs, and wherein each row of reservoirs comprises an identical bioactive agent.

Aspect 18. The device of any one of aspects 1-17, further comprising a plurality of distances between reservoirs, wherein the plurality of distances are measured parallel to the longitudinal axis.

Aspect 19. The device of aspect 18, wherein each distance of the plurality of distances is the same.

Aspect 20. The device of aspect 18, wherein at least a first one of the plurality of distances is different from at least a second one of the plurality of distances.

Aspect 21. The device of any one of aspects 1-20, wherein at least a first one of the plurality of reservoirs is adjacent to a different second biodegradable polymer from at least a second one of the plurality of reservoirs.

Aspect 22. The device of any one of aspects 1-20, wherein each one of the plurality of reservoirs is adjacent to the same second biodegradable polymer.

Aspect 23. The device of any one of aspects 1-22, wherein each one of the plurality of reservoirs comprises the same volume.

Aspect 24. The device of any one of aspects 1-22, wherein at least a first one of the plurality of reservoirs comprises a different volume from at least a second one of the plurality of reservoirs.

Aspect 25. The device of any one of aspects 1-24, wherein the at least one second biodegradable polymer includes an additional bioactive agent.

Aspect 26. The device of aspect 25, wherein the additional bioactive agent is the same as the one or more bioactive agents in the plurality of reservoirs.

Aspect 27. The device of aspect 25, wherein the additional bioactive agent is different from the one or more bioactive agents in the plurality of reservoirs.

Aspect 28. A method for pulsed delivery of one or more bioactive agents to a subject, the method comprising implanting a device according to any one of aspects 1-27 in the subject.

Aspect 29. The method of aspect 28, wherein the device is implanted by injection.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Device Optimization and Drug Release Profiles

### Correlation Between Polymer Mass and Thickness of Polymer Films

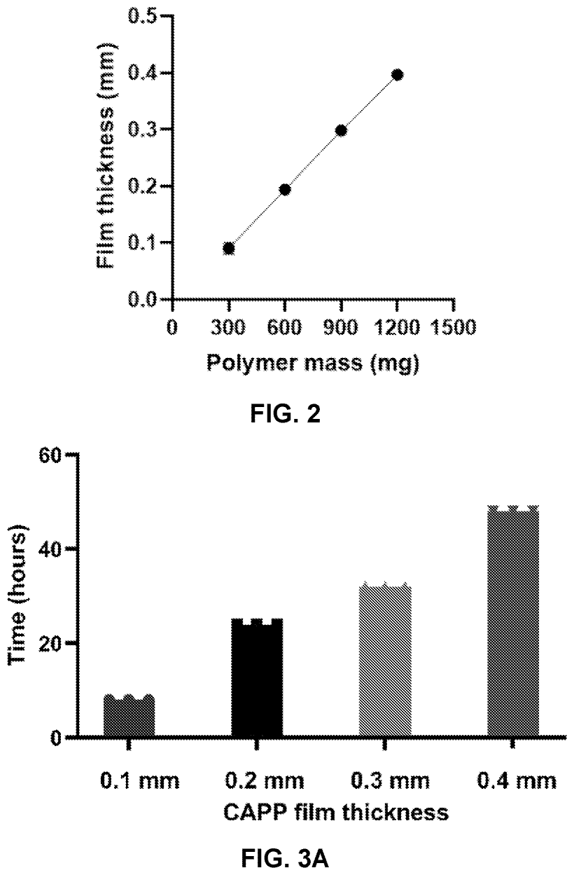

The optimum conditions for formation of surface-eroding Cellulose Acetate Phthalate-Pluronic F-127 (CAPP) films of varying thickness was established. After screening a variety of conditions (different mol ratios of CAP to P, solvent choice, evaporation conditions, and polymer weight percent), it was determined that CAP:P mixtures dispersed well at 7% w/v in acetone and 70:30 mol ratio CAP:P films evaporated from acetone at 4° C. to form uniform and rigid films. Using Teflon (PTFE)-coated dishes with uniform diameter, films from 300, 600, 900, and 1200 mg of CAPP polymer mixture were produced. These produced films of ˜0.1, 0.2, 0.3, and 0.4 mm, respectively (FIG. 2). Thus, there was a strong linear correlation between polymer mass and thickness of the films, enabling fabrication of consistent films moving forward.

### Established Erosion Time of Polymer Films and the Correlation Between Film Thickness and Erosion Time

The next goal—knowing the predictable thicknesses of films based on polymer weight—was to establish any correlation between film thickness and erosion time. First, it was confirmed that the CAPP films do degrade via surface erosion, as they maintain similar mechanical properties and decrease in size as degradation occurs. Moreover, erosion time was dependent on the thickness of the films, where 0.1, 0.2, 0.3, and 0.4 mm thick films were fully eroded at 36, 48, 66, and 84 hrs, respectively.

Quantified Drug Release from Films of Varying Thickness Using Fluorescent Dyes as Drug Surrogates

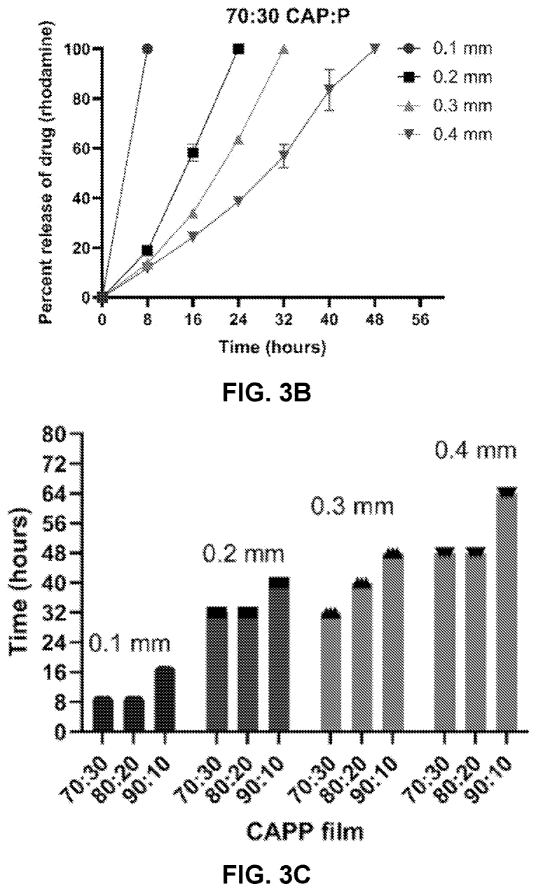

Lastly, drug release from single-layered CAPP films of 0.1, 0.2, 0.3, and 0.4 mm thickness was quantified. Erosion time was dependent on the thickness of the films, where 0.1, 0.2, 0.3, and 0.4 mm thick films were fully eroded at 8, 24, 36, and 48 hrs, respectively (FIG. 3A). Drug release was measured by encapsulating rhodamine fluorescent dye within the films and closely matched the erosion times reported above. Release from 0.1 mm thick films was completed by ˜8 hrs (FIG. 3B). Release from 0.2 mm thick films was completed by ˜20 hrs (FIG. 3B). Release from 0.3 mm thick films was completed by ˜28 hrs (FIG. 3B). Release from 0.4 mm thick films was completed by ˜44 hrs (FIG. 3B). Surprisingly, although the films eroded uniformly via surface erosion. Release times were similar for 70:30 and 80:20 CAP:P films in most cases but slightly longer for 90:10 CAP:P films (FIG. 3C).

### Example 2: Device Fabrication

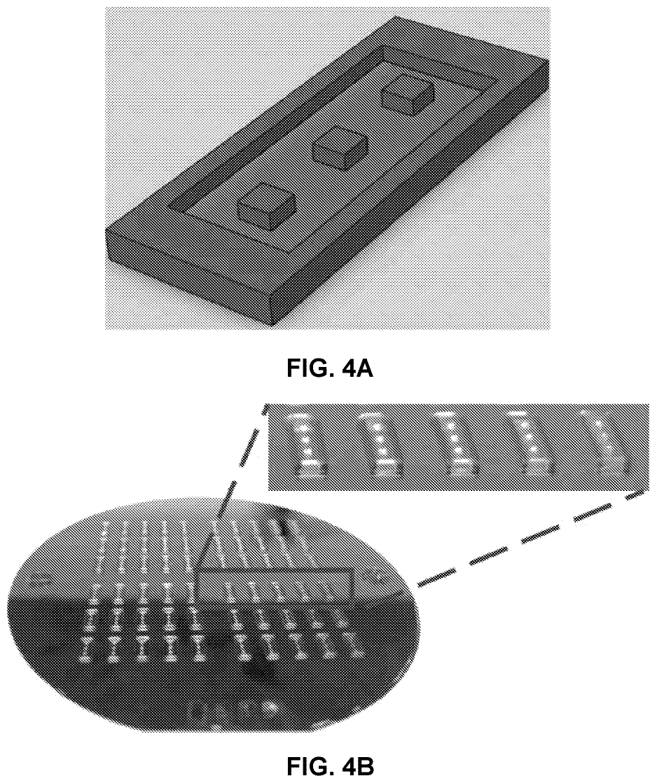

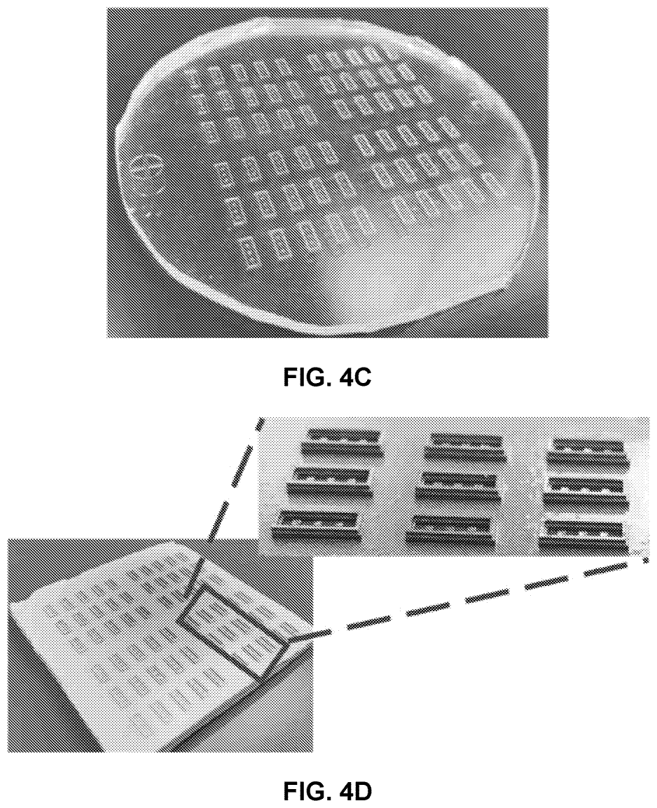

The mold design for the μfuse device is shown in FIG. 4A. Fabrication began with the creation of a photolithographically-defined mold. Briefly, SU-8 photoresist was spun onto a silicon wafer and the negative shape of the μfuse device was defined using a high-resolution photomask. The outer border of the μfuse device mold was taller than the three reservoir posts in the middle for successful polymer molding later in the process. Therefore, the master fabrication required two layers of photoresist and two exposure steps, which lengthened the time required for successful process optimization and device fabrication. The three square posts in the middle each have dimensions of 200 μm×200 μm×200 μm. The outer wall is 100 μm thick and 400 μm tall. FIG. 4B shows the completed SU-8 negative mold on a 3 inch silicon wafer. Next, a replica of the mold was created by using the two-part elastomer polydimethylsiloxane (PDMS). PDMS monomer and curing agent were mixed in a 10:1 ratio, poured over the SU-8 mold, and allowed to cure (FIG. 4C). The final mold-making step was to use the PDMS replica as a mold for the two-part thermally conductive epoxy RenCast 4037. After several optimization, an epoxy mold (FIG. 4D) was successfully created. An epoxy mold is necessary for later hot embossing steps because it is more mechanically robust and will last for more impressions.

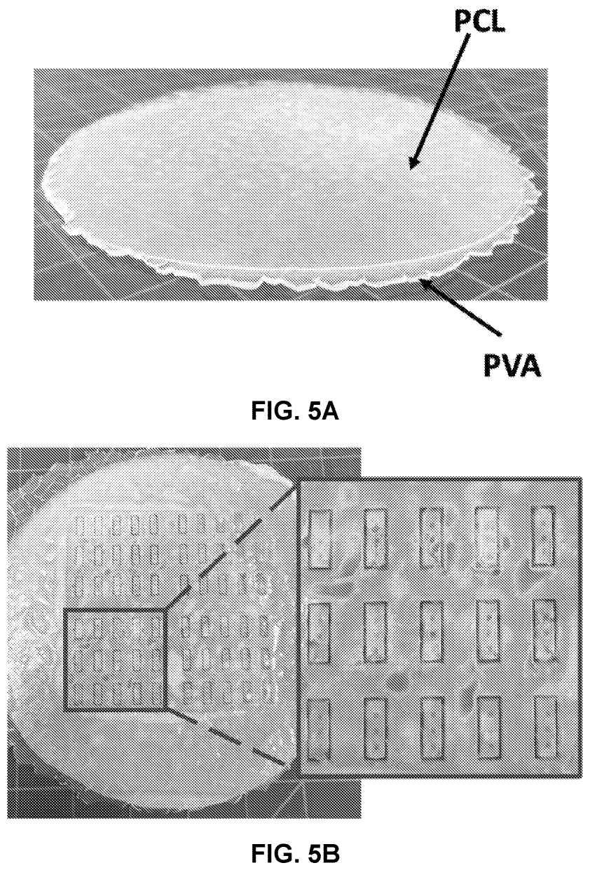

Once an epoxy mold suitable for hot embossing had been fabricated, parameter were optimized for creating thin films of polyvinylalcohol (PVA) and polycaprolactate (PCL). These polymers form the base layer of the final μfuse drug delivery device. First, PVA pellets were compressed in a heated press at 1200 PSI and 154° C. for 360 sec and then cooled under pressure for 2 hrs. This created a film of PVA approximately 1 mm thick. The pressure was released on the PVA film and PCL pellets were placed on top. The pellets and film were then subjected to 200 PSI at 71° C. for 120 sec and cooled under pressure for 30 min. This procedure yielded repeatable films of the appropriate thicknesses to fabricate fuse devices (˜400 μm for PCL and ˜1 mm for PVA) (FIG. 5A). After the films were fabricated, hot embossing conditions were optimized. Air bubbles, mold adhesion to the thin films, and poor feature reproduction were addressed during optimization. μfuse devices were successfully hot embossed by assembling the PCL/PVA film and epoxy mold within the heated press and applying a pressure of 55 PSI at 57° C. for 5 min. The mold and films were then cooled to 4° C. and carefully demolded. Initial experiments yielded ˜10% of usable μfuse devices after hot embossing.

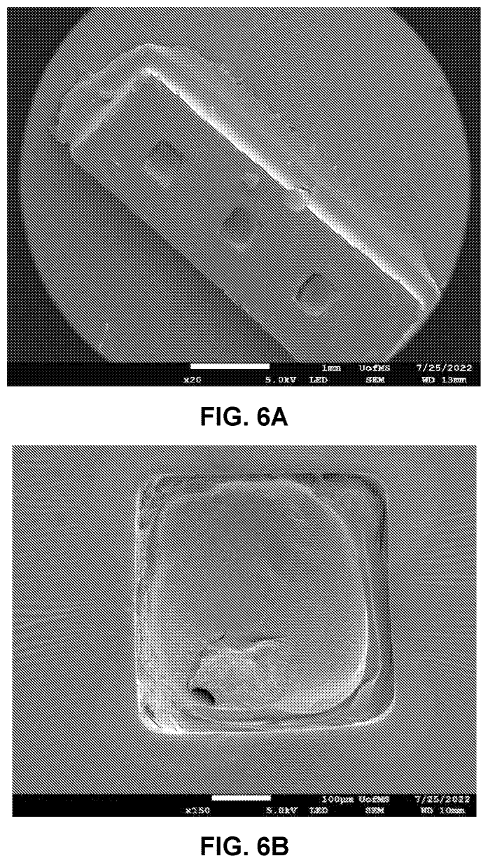

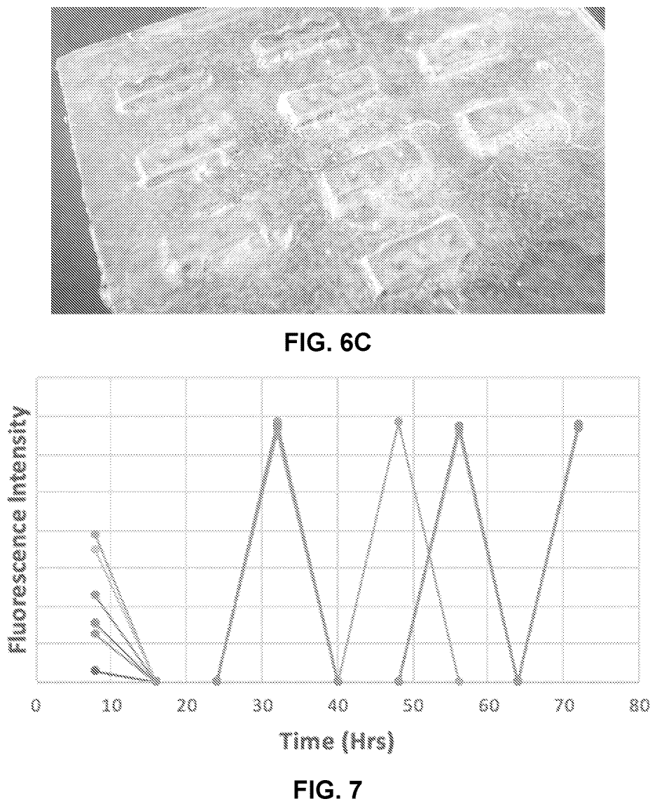

FIGS. 6A-6C depict devices that have been fully loaded with fluorescein (FITC) as a model drug compound, coated with a CAPP film, and then encapsulated on all sides in an outer layer of PCL. FIG. 6A shows a scanning electron microscope (SEM) image of one device with three FITC-loaded drug reservoirs prior to CAPP fuse application. FIG. 6B shows the same, but zoomed in to a single reservoir where it can be seen that FITC is completely filling the reservoir. FIG. 6C shows an array of multiple devices that have been coated with CAPP fuses, followed by a final encapsulation in PCL to form the complete, functional devices.

FIG. 7 depicts a release study (FITC from the reservoirs pictured in FIGS. 6A-6C) for 6 separate devices. Measurements were taken every 8 hours until all three reservoirs had released their loaded FITC. The fluorescence intensity observed at the first read was not representative of the desired release profile; this was a result of residual FITC on and in the device. This residual is the result of a less than perfect loading protocol which is currently being optimized. However, the devices did demonstrate clear and fairly precise intermittent release of the FITC dye with an average time between reservoir release of approximately 20 hours.

### Example 3: Microfabrication and In Vitro Validation of a μFuse Device Enabling Delivery of 5HT2A Agonists

### Fabrication and Structural Characterization

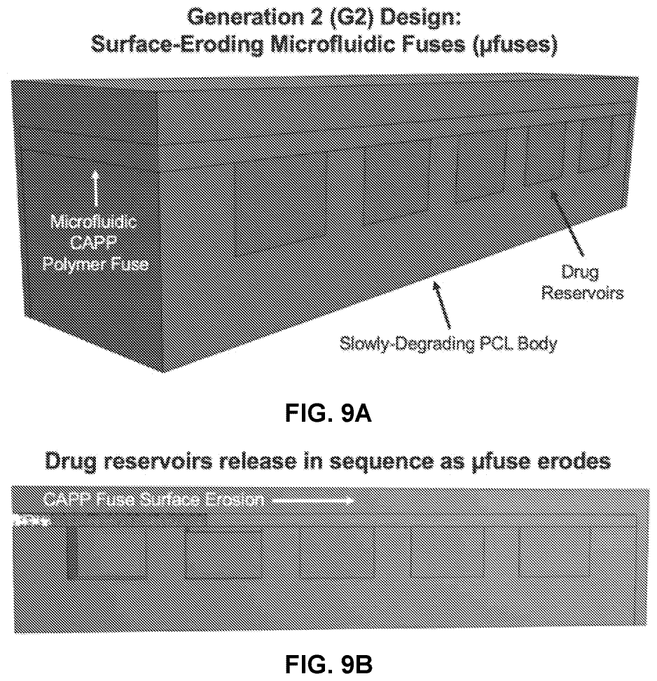

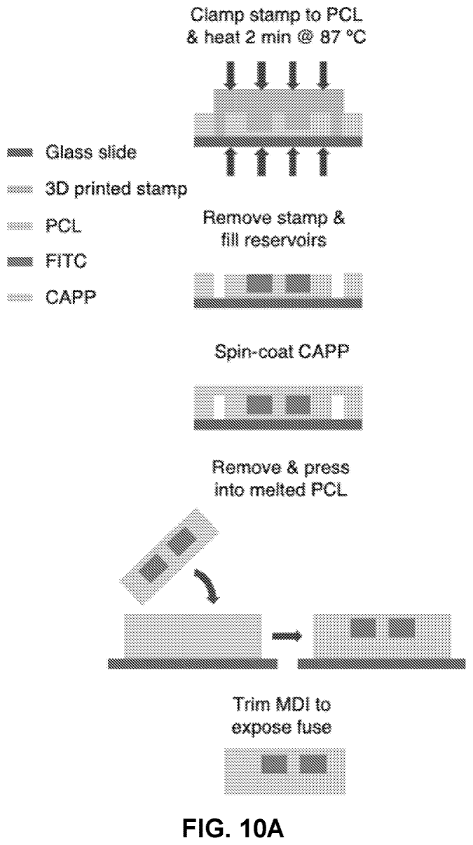

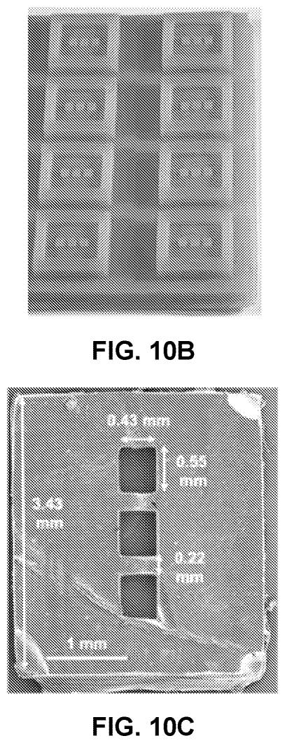

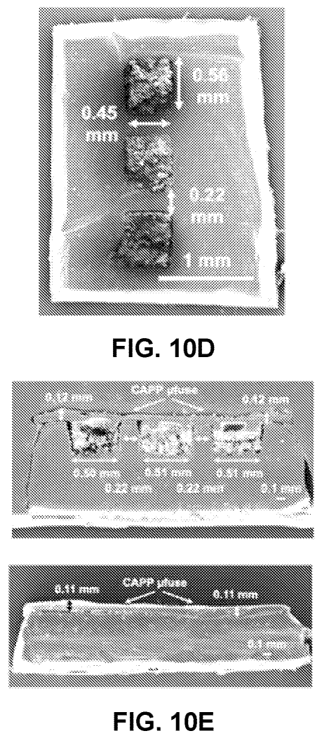

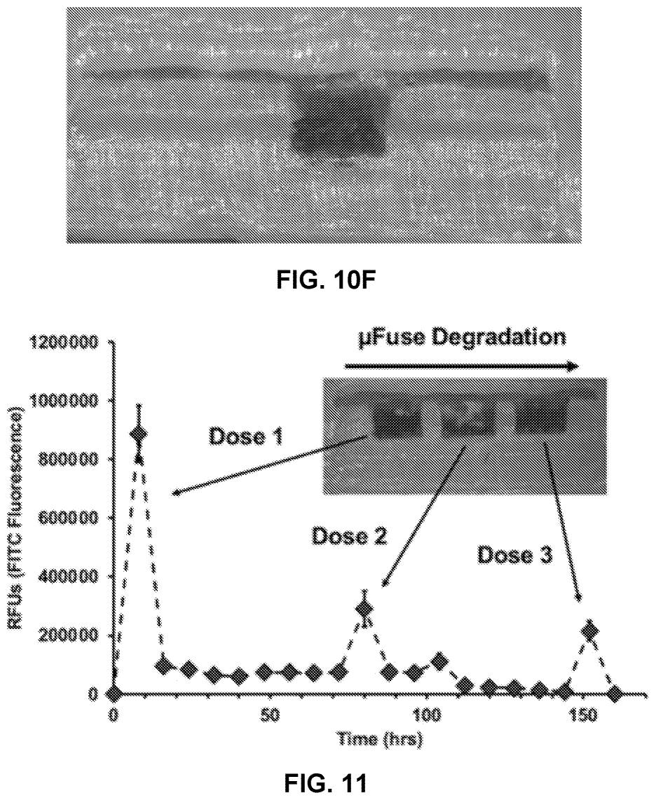

A first-generation prototype for interval microdosing of psychedelics was developed using layered CAPP films. While multilayered CAPP films provide initial proof-of-concept for a biomaterials-based MDI, they suffer from a few major shortcomings that will limit their clinical translation. Therefore, a second-generation biomaterials-based MDI has been developed and was pursued further in these studies (FIGS. 9A-9B). The primary innovation that makes this technology distinct is the use of a surface-eroding, biodegradable μfuse that is used to program the interval of drug release from reservoirs beneath the fuse. FIGS. 10A-10F show the microfabrication process by which the μfuse-based devices are generated. Briefly, a UV-crosslinked resin stamp is produced with a high-resolution Phrozen 8K digital light processing (DLP) 3D printer. The stamp is then placed on top of a glass slide coated with a poly-ε-caprolactone (PCL) film and the assembly is clamped together and placed in an oven at 87° C. for two minutes. After the assembly is removed and allowed to cool, the stamp is removed and FITC powder is manually packed into the reservoirs with a weigh spatula. The devices are then mounted in a spin-coater and successive thin layers of CAPP are spun on top of the devices until a CAPP layer of sufficient thickness covers the reservoirs. Finally, the devices are pressed CAPP side down into a melted PCL film to encapsulate the fuse and coat the device with PCL. After cooling, one end of the device is trimmed to expose the μfuse and allow for unidirectional erosion of the fuse. SEM images show the successful fabrication of second-generation MDIs; Positive Stamp (FIG. 10B), Stamped PCL (FIG. 10C), FITC-Filled PCL (FIG. 10D), CAPP-Coated PCL (FIG. 10E), and Final Encapsulated Structure drug reservoir cross-section (FIG. 10F).

In Vitro Precise Interval Drug Delivery from μFuse-Based MDIs

Once microfabrication of the μfuse-based implants was accomplished, tests were performed to see if precise intervals of drug release could be achieved from the implants. Fluorescein (FITC) was loaded into the reservoirs of a three-dose implant designed to release the drug every three days (Q72), after which μfuse degradation was monitored via imaging and FITC release on a microplate reader. Linear degradation of CAPP μfuses was observed over time which ensures reliable, timed release of reservoirs in sequence as they are ‘uncovered’ by μfuse degradation. Importantly, FITC release tracked with the μfuse degradation as well; all three reservoirs released in sequence, each pulse was evenly spaced three days apart as designed, and each pulse was rapid and of equivalent magnitude (FIG. 11).

In Vivo Release of μFuse Devices Loaded with 5HT2A Agonist Microdoses

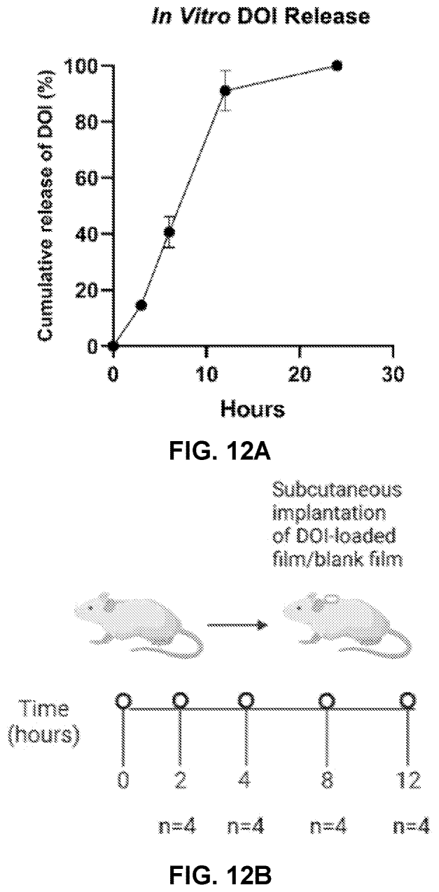

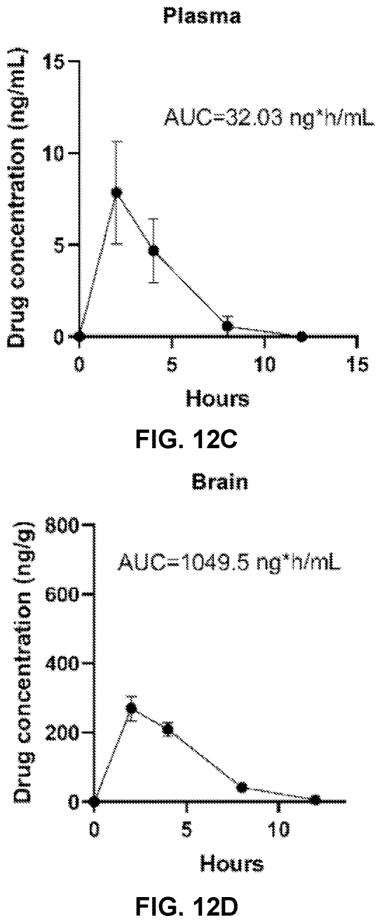

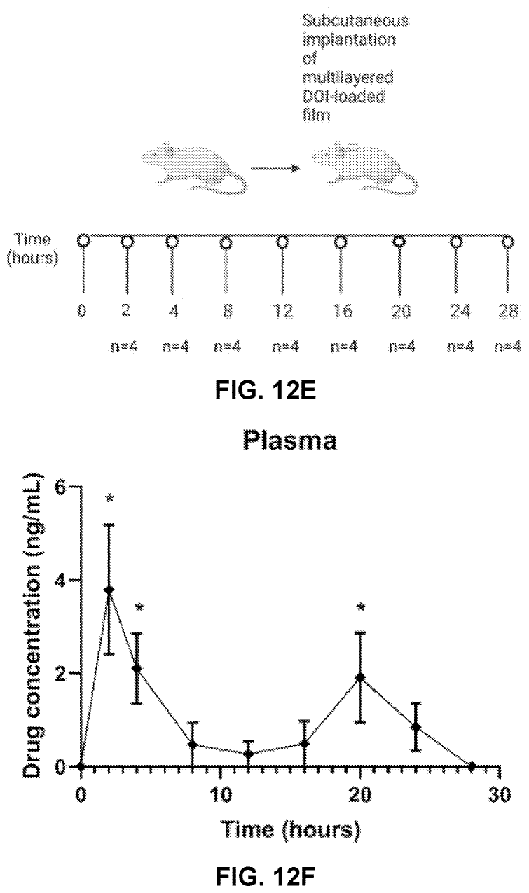

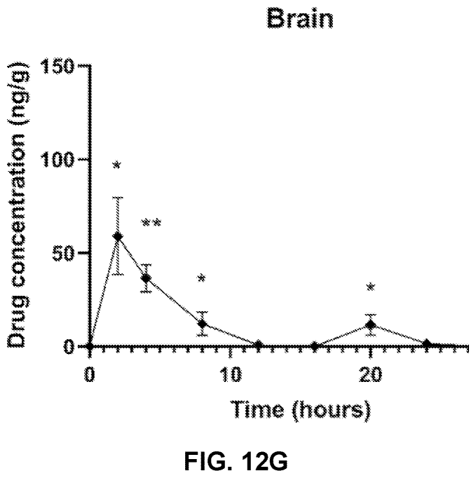

To confirm that 5HT_{2A} agonists could be effectively delivered in vivo from an implantable drug delivery system, the encapsulation and release of DOI (2,5-dimethoxy-4-iodoamphetamine) from CAPP films was investigated. DOI release was first observed from single-layered CAPP films. Importantly, the release kinetics of DOI closely mimicked that of rhodamine and FITC which were used as model drugs in initial studies (FIG. 12A). The DOI-loaded, single-layered films were next implanted subcutaneously in CD-1 mice (50:50 M:F mice) and monitored the pharmacokinetics and biodistribution of DOI after implantation (FIGS. 12B-12D). It was found that DOI was rapidly absorbed from the subcutaneous films with a peak plasma concentration at 2 hrs post-implantation, after which it was cleared from circulation, closely mimicking the published pharmacokinetic profiles of the drug (FIG. 12C). Moreover, DOI rapidly accumulated in the brain after which it was cleared with similar kinetics to the blood plasma (FIG. 12D). After this initial optimization, multilayered CAPP films with DOI encapsulated in the ‘release’ layers were generated and implanted them subcutaneously in CD-1 mice (50:50 M:F mice; FIG. 12E). Again, rapid absorption of DOI and accumulation in the brain (as well as kidneys and liver) from the first ‘release’ layer was observed (FIGS. 12F-12G). After DOI levels returned to baseline, a second distinct pulse of DOI in the plasma and organs was observed ˜20 hours after the first pulse. These results represent the first successful interval dosing of psychedelics from a biomaterial and confirm the ability to assess the encapsulation and interval release of psychedelics from an implantable drug delivery system.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Hossain M, et al. Interval delivery of 5HT(2A) agonists using multilayered polymer films. J Biomed Mater Res A. June 2023; 111(6):790-800. doi:10.1002/jbm.a.37497
2. de la Fuente Revenga M, et al. Fully automated head-twitch detection system for the study of 5-HT(2A) receptor pharmacology in vivo. Sci Rep. Oct. 3 2019; 9(1):14247. doi:10.1038/s41598-019-49913-4

1. A device for delivering one or more bioactive agents to a subject, the device comprising
(a) a first layer comprising a first biodegradable polymer, wherein the first layer has a first side and a second side, and wherein a plurality of reservoirs comprising the one or more bioactive agents are present on the first side of the first layer;
(b) a second layer comprising at least one second biodegradable polymer, wherein the second layer is adjacent to the first side of the first layer;
(c) a longitudinal axis; and
(d) a third layer comprising a third biodegradable polymer, wherein the third layer encapsulates the first layer and the second layer, and wherein at least one face of the device perpendicular to the longitudinal axis comprises an exposed second layer surface not coated by the third biodegradable polymer.

2. The device of claim 1, wherein the first biodegradable polymer and the at least one second biodegradable polymer are different polymers.

3. The device of claim 1, wherein the first biodegradable polymer and the third biodegradable polymer are the same polymer.

4. The device of claim 1, wherein the first biodegradable polymer comprises poly-ε-caprolactone (PCL), polysebacic acid, polylactic acid, a polyester, a polyanhydride, a polyurethane, a polyphosphazine, a polyalkylcyanoacrylate, a polypeptide, a block copolymer comprising polyethylene glycol (PEG), or any combination thereof.

5. The device of claim 1, wherein the first layer has a thickness of from about 0.1 mm to about 2 mm.

6. (canceled)

7. The device of claim 1, wherein the at least one second biodegradable polymer comprises a mixture of cellulose acetate phthalate (CAP) and a poloxamer, a polyanhydride, a polyorthoester, or any combination thereof.

8. The device of claim 1, wherein the at least one second biodegradable polymer comprises a mixture of cellulose acetate phthalate (CAP) in the amount of about 50 mol % to about 90 mol % and a poloxamer in the amount of about 10 mol % to about 50 mol %.

9. The device of claim 1, wherein the second layer has a thickness of from about 0.1 mm to about 2 mm.

10. The device of claim 1, wherein the third biodegradable polymer comprises poly-ε-caprolactone (PCL), polysebacic acid, polylactic acid, a polyester, a polyanhydride, a polyurethane, a polyphosphazine, a polyalkylcyanoacrylate, a polypeptide, a block copolymer comprising polyethylene glycol (PEG), or any combination thereof.

11. The device of claim 1, wherein the third layer has a thickness of from about 0.1 mm to about 2 mm.

12. The device of claim 1, wherein the one or more bioactive agents comprises an antibiotic, a pain reliever, an immune modulator, a growth factor, an enzyme inhibitor, a hormone, a messenger molecule, a cell signaling molecule, a receptor agonist, an oncolytic virus, a chemotherapy agent, a receptor antagonist, a nucleic acid, a nanoparticle, a drug conjugate, an antibody-drug conjugate, or any combination thereof.

13. The device of claim 1, wherein each one of the plurality of reservoirs comprises a different bioactive agent.

14. The device of claim 1, wherein each one of the plurality of reservoirs comprises the same bioactive agent.

15. The device of claim 1, wherein the plurality of reservoirs are arranged in one or more rows parallel to the longitudinal axis.

16.-17. (canceled)

18. The device of claim 1, further comprising a plurality of distances between reservoirs, wherein the plurality of distances are measured parallel to the longitudinal axis.

19.-20. (canceled)

21. The device of claim 1, wherein at least a first one of the plurality of reservoirs is adjacent to a different second biodegradable polymer from at least a second one of the plurality of reservoirs.

22. The device of claim 1, wherein each one of the plurality of reservoirs is adjacent to the same second biodegradable polymer.

23.-24. (canceled)

25. The device claim 1, wherein the at least one second biodegradable polymer includes an additional bioactive agent.

26-27. (canceled)

28. A method for pulsed delivery of one or more bioactive agents to a subject, the method comprising implanting a device according to claim 1 in the subject.

29. The method of claim 28, wherein the device is implanted by injection.
